SEQUENCE LISTING (20 sequences)

SEQ ID NO: 1 - RNA, 19 nt, Artificial Sequence
  Description of Artificial Sequence Synthetic oligonucleotide
gcaacguccugcugguuaa

SEQ ID NO: 2 - RNA, 19 nt, Artificial Sequence
  Description of Artificial Sequence Synthetic oligonucleotide
ccucauagcugacauugaa

SEQ ID NO: 3 - RNA, 19 nt, Artificial Sequence
  Description of Artificial Sequence Synthetic oligonucleotide
gaacuguacgcacucauga

SEQ ID NO: 4 - RNA, 19 nt, Artificial Sequence
  Description of Artificial Sequence Synthetic oligonucleotide
ggagaucccugugagcaau

SEQ ID NO: 5 - RNA, 19 nt, Artificial Sequence
  Description of Artificial Sequence Synthetic oligonucleotide
caauuagaaucccucaaua

SEQ ID NO: 6 - RNA, 19 nt, Artificial Sequence
  Description of Artificial Sequence Synthetic oligonucleotide
guauaaggcuugcaacuug

SEQ ID NO: 7 - RNA, 19 nt, Artificial Sequence
  Description of Artificial Sequence Synthetic oligonucleotide
gacauuaccucuucagcuu

SEQ ID NO: 8 - RNA, 19 nt, Artificial Sequence
  Description of Artificial Sequence Synthetic oligonucleotide
cgacuucgcugugaauuag

SEQ ID NO: 9 - RNA, 19 nt, Artificial Sequence
  Description of Artificial Sequence Synthetic oligonucleotide
gcgcagcggucucaucuac

SEQ ID NO: 10 - RNA, 19 nt, Artificial Sequence
  Description of Artificial Sequence Synthetic oligonucleotide
agucauauucugcguagga

SEQ ID NO: 11 - RNA, 19 nt, Artificial Sequence
  Description of Artificial Sequence Synthetic oligonucleotide
gcaacgggcugucggucaa

SEQ ID NO: 12 - RNA, 19 nt, Artificial Sequence
  Description of Artificial Sequence Synthetic oligonucleotide
ggugauuguucaggucgaa

SEQ ID NO: 13 - RNA, 19 nt, Artificial Sequence
  Description of Artificial Sequence Synthetic oligonucleotide
gagcgugccuucugagaga

SEQ ID NO: 14 - RNA, 19 nt, Artificial Sequence
  Description of Artificial Sequence Synthetic oligonucleotide
ggagaugguuccaguuauc

SEQ ID NO: 15 - RNA, 19 nt, Artificial Sequence
  Description of Artificial Sequence Synthetic oligonucleotide
aaucacaucuaucagcaaa

SEQ ID NO: 16 - RNA, 19 nt, Artificial Sequence
  Description of Artificial Sequence Synthetic oligonucleotide
agacauaggagaugauuca

SEQ ID NO: 17 - DNA, 20 bp, Artificial Sequence
  Description of Artificial Sequence Synthetic oligonucleotide
agatgcaaccaatcctgctt

SEQ ID NO: 18 - RNA, 18 nt, Artificial Sequence
  Description of Artificial Sequence Synthetic oligonucleotide
caugucccccgaugaucu

SEQ ID NO: 19 - DNA, 20 bp, Artificial Sequence
  Description of Artificial Sequence Synthetic oligonucleotide
gaaggtgaaggtcggagtca

SEQ ID NO: 20 - DNA, 22 bp, Artificial Sequence
  Description of Artificial Sequence Synthetic oligonucleotide
ggggtcattgatggcaacaata

(19) United States
(12) Patent Application Publication
(10) Pub. No.: US 2024/0216354 A1
(43) Pub. Date: Jul. 4, 2024

(54) IMMUNOMODULATORY IMIDE DRUGS AS ZETA-CHAIN-ASSOCIATED PROTEIN KINASE 70 (ZAP70) AGONISTS AND USES THEREOF

(71) Applicant: Dana-Farber Cancer Institute, Inc., Boston, MA (US)

(72) Inventors: Teru Hideshima, Brookline, MA (US); Kenneth C. Anderson, Wellesley, MA (US)

(73) Assignee: Dana-Farber Cancer Institute, Inc., Boston, MA (US)

(21) Appl. No.: 17/766,189

(22) PCT Filed: Oct. 1, 2020
(86) PCT No.: PCT/US2020/053719

§ 371 Date: Apr. 1, 2022

Related U.S. Application Data
(60) Provisional application No. 62/986,605, filed on Mar. 6, 2020.
(60) Provisional application No. 62/911,104, filed on Oct. 4, 2019.

(51) Int. Cl. A61K 31/454; A61K 31/573; A61K 31/69

(52) U.S. Cl. CPC A61K 31/454; A61K 31/573; A61K 31/69

(57) ABSTRACT

The present disclosure provides methods of treating a disease in a subject in need thereof (e.g., proliferative diseases (e.g., cancer (e.g., multiple myeloma))) comprising administering to a subject in need thereof an effective amount of an immunomodulatory drug (e.g., pomalidomide, thalidomide, lenalidomide, iberdomide). The disclosed IMiDs may increase the activity of a kinase (e.g., Zap-70). Also provided are kits comprising the disclosed IMiDs.

### RELATED APPLICATIONS

This application claims priority under 35 U.S.C. § 119(e) to U.S. Provisional Patent Application No. 62/911,104, filed Oct. 4, 2019, and U.S. Provisional Patent Application No. 62/986,605, filed Mar. 6, 2020, which are hereby incorporated by reference in their entireties.

### GOVERNMENT SUPPORT

This invention was made with government support under Grant Numbers P50 CA100707, R01-CA050947, and R01-CA178264 awarded by the National Institutes of Health. The government has certain rights in the invention.

### BACKGROUND

Among the most important treatment advances in multiple myeloma (MM) is the development of the immunomodulatory drugs (IMiDs) thalidomide, lenalidomide, and pomalidomide. Their multiple anti-MM effects include: induction of growth arrest and apoptosis in tumor cells; downregulation of adhesion molecules and MM cell binding to cellular components and extracellular matrix proteins in the bone marrow (BM); anti-angiogenesis; modulation of cytokines; and immunomodulation associated with enhanced T cell, NK cell, and NK-T cell activity, along with decreased regulatory T cell activity (Hideshima T., et al.; Blood 2000, 96, 2943-2950; Mitsiades N., et al.; Blood 2002, 99, 4525-4530; Anderson K. C., et al.; J Natd Compr Canc Netw. 2016, 14, 493-496). Multiple groups have shown that thalidomide, lenalidomide, and pomalidomide directly bind to cereblon (CRBN), forming an E3 ubiquitin ligase complex with damaged DNA binding protein 1 (DDB1), cullin-4A, and regulator of cullinsl (Ito T., et al.; Science 2010, 327, 1345-1350; Lopez-Girona A., et al., Leukemia 2012, 26, 2326-2335.), thereby triggering proteasomal degradation of Ikaros (IKZF1) and Aiolos (IKZF3) followed by downregulation of interferon regulatory factor 4 (IRF4) and MM cell growth (Kronke J., et al., Science 2014, 343, 301-305; Lu G., et al. Science 2014, 343, 305-309). Pomalidomide was shown to directly binds to TP53 regulating kinase (TP53RK) and inhibits its activity, which is associated with significant MM cell growth inhibition via both p53 dependent and independent pathways (Hideshima T., Blood 2017, 129, 1308-1319).

Studies have also begun to delineate the molecular mechanisms whereby IMiDs mediate their immune effects. For example, lenalidomide triggers CD28 tyrosine phosphorylation in T cells, followed by NF-κB activation (LeBlanc R., et al., Blood 2004, 103, 1787-1790). IMiDs induce IL-2 and γ-interferon, while inhibiting suppressor of cytokine signaling, in CD4+ T-cells, CD8+ T-cells, and natural-killer (NK) T cells from both BM and peripheral blood (PB) of MM patients (Gorgun, G., et al. Blood 2010, 116, 3227-3237). This upregulation of immune activity by pomalidomide and lenalidomide is, at least in part, mediated by their binding to CRBN and triggering degradation of T-cell repressors IKZF1 and IKZF3, thereby allowing for increased transcription and secretion of cytokines including IL-2 (Gandhi A. K., et al., Br J Haematol. 2014, 164, 811-821.). It has been demonstrated that IL-2-primed PB mononuclear cells (PBMCs) treated with IMiDs showed significantly increased lysis of MM cell lines, which was not major histocompatibility complex-class restricted (Davies F. E., et al., Blood 2001, 98, 210-216). It has also been reported that IMiDs enhance both NK cell and NK-T cell cytotoxicity and antibody-dependent cellular cytotoxicity (ADCC), at least in part due to triggering IL-2 production from T cells (Hayashi T., et al., Br J Haematol. 2005, 128, 192-203; Chang D. H., et al., Blood 2006, 108, 618-621; Reddy N., et al., Br J Haematol. 2008, 140, 36-45; Wu L., et al., Clin Cancer Res. 2008, 14, 4650-4657; Richter J., et al., Blood 2013, 121, 423-430; Pittari G., et al., Front Immunol. 2017, 8, 1444.). Moreover, a recent study has shown that lenalidomide can enhance secretion of IFN-γ and GZM-B from antigen-specific T-cells (Neuber B., et al., Oncotarget. 2017, 8, 98200-98214).

### SUMMARY OF THE INVENTION

To date, the molecular mechanisms whereby IMiDs induce NK cell cytotoxicity have not been elucidated. In the present disclosure, the role of zeta-chain-associated protein kinase-70 (Zap-70), in mediating the increased NK cell cytotoxicity triggered by IMiDs was characterized. Zap-70 is a 70 kDa cytoplasmic protein tyrosine kinase composed of two SH2 domains and a carboxy-terminal kinase domain initiating T-cell responses by the antigen receptor (Wang H., et al., Cold Spring Harb Perspect Biol. 2010, 2, a002279). It was found that IMiDs directly bind and activate Zap-70. Increased GZM-B expression and NK cell activity triggered by IMiDs is associated with Zap-70 activation, which was inhibited by Zap-70 knockdown, independent of CRBN. A second mechanism whereby IMiDs trigger GZMB and NK cytotoxicity is CRBN- and IKZF3-mediated and can be inhibited by knockdown of CRBN or IKZF-3, independent of Zap-70. Thus, IMiDs can enhance NK and T cell cytotoxicity in ZAP-70-mediated CRBN independent, as well as CRBN-mediated ZAP-70 independent mechanisms.

The current disclosure is based, in part, on the discovery that the IMiDs disclosed herein may increase the activity of a kinase (e.g., Zap-70), and in certain embodiments, the IMiDs may be specific or selective for Zap-70 over one or more other kinases. Provided herein are methods of using the provided IMiDs and kits comprising the IMiDs (e.g., for treating a disease in a subject in need thereof, or increasing the activity of a kinase in a subject in need thereof, a biological sample, or a cell).

In certain embodiments, the disease is a proliferative disease. In certain embodiments, the proliferative disease is cancer. In certain embodiments, the cancer is multiple myeloma.

Another aspect of the present disclosure relates to methods of increasing the activity of a kinase using an IMiD in a biological sample or subject in need thereof.

The present invention provides methods for administering to a subject in need thereof an effective amount of an IMiD (e.g., thalidomide, pomalidomide, lenalidomide, iberdomide). In certain embodiments, the IMiD is a small molecule. Also described are methods for contacting a biological sample or cell with an effective amount of an IMiD, or a pharmaceutically acceptable salt, solvate, hydrate, polymorph, co-crystal, tautomer, stereoisomer, isotopically labeled derivative, or prodrug thereof. In certain embodiments, a method described herein further includes administering to the subject in need thereof an additional pharmaceutical agent. In certain embodiments, a method described herein further includes contacting the biological sample or cell with an additional pharmaceutical agent. In certain embodiments, the additional pharmaceutical agent is a chemotherapeutic agent (e.g., bortezomib).

In yet another aspect, the present invention provides IMiDs, and pharmaceutically acceptable salts, solvates, hydrates, polymorphs, co-crystals, tautomers, stereoisomers, isotopically labeled derivatives, and prodrugs thereof, for use in the treatment of a disease (e.g., a proliferative disease, such as cancer) in a subject in need thereof.

In yet another aspect, the present invention provides IMiDs, and pharmaceutically acceptable salts, solvates, hydrates, polymorphs, co-crystals, tautomers, stereoisomers, isotopically labeled derivatives, and prodrugs thereof, for use in the prevention of a disease (e.g., a proliferative disease, such as cancer) in a subject in need thereof.

In another aspect, the present disclosure provides uses of IMiDs, and pharmaceutically acceptable salts, solvates, hydrates, polymorphs, co-crystals, tautomers, stereoisomers, isotopically labeled derivatives, and prodrugs thereof, in the manufacture of a medicament for treating a disease in a subject in need thereof.

In another aspect, the present disclosure provides uses of IMiDs, and pharmaceutically acceptable salts, solvates, hydrates, polymorphs, co-crystals, tautomers, stereoisomers, isotopically labeled derivatives, and prodrugs thereof, in the manufacture of a medicament for preventing a disease in a subject in need thereof.

In another aspect, the present disclosure provides kits comprising: an immunomodulatory drug, or a pharmaceutically acceptable salt, solvate, hydrate, polymorph, co-crystal, tautomer, stereoisomer, isotopically labeled derivative, or prodrug thereof; and instructions for using the immunomodulatory drug, or a pharmaceutically acceptable salt, solvate, hydrate, polymorph, co-crystal, tautomer, stereoisomer, isotopically labeled derivative, or prodrug thereof.

The details of one or more embodiments of the present disclosure are set forth herein. Other features, objects, and advantages of the present disclosure will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

IMiDs described herein can comprise one or more asymmetric centers, and thus can exist in various isomeric forms, e.g., enantiomers and/or diastereomers. For example, the IMiDs described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC), supercritical fluid chromatography (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses IMiDs described herein as individual isomers substantially free of other isomers, and alternatively, as mixtures of various isomers.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences (1977) 66:1-19. Pharmaceutically acceptable salts of the IMiDs described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

The term “solvate” refers to forms of the IMiDs that are associated with a solvent, usually by a solvolysis reaction. This physical association may include hydrogen bonding. Conventional solvents include water, methanol, ethanol, acetic acid, DMSO, THF, diethyl ether, and the like. The IMiDs may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to an IMiD that is associated with water. Typically, the number of the water molecules contained in a hydrate of a IMiD is in a definite ratio to the number of the IMiD molecules in the hydrate. Therefore, a hydrate of an IMiD may be represented, for example, by the general formula R·x H₂O, wherein R is the IMiD and wherein x is a number greater than 0. A given IMiD may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” refer to compounds (e.g., IMiDs) that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of an IMiD of interest.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorphs” refers to a crystalline form of an IMiD (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of an IMiD can be prepared by crystallization under different conditions.

The term “prodrugs” refer to IMiDs, which have cleavable groups and become by solvolysis or under physiological conditions the IMiD which is pharmaceutically active in vivo. Such examples include, but are not limited to, ester derivatives and the like. Other derivatives of the IMiDs have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of a parent acid compound with a substituted or unsubstituted amine, or acid anhydrides, or mixed anhydrides. Simple aliphatic or aromatic esters, amides, and anhydrides derived from acidic groups pendant on the IMiDs are particular prodrugs. In some cases it is desirable to prepare double ester type prodrugs such as (acyloxy)alkyl esters or ((alkoxycarbonyl)oxy)alkylesters. C₁ to C₈ alkyl, C₂-C₈ alkenyl, C₂-C₈ alkynyl, aryl, C₇-C₁₂ substituted aryl, and C₇-C₁₂ arylalkyl esters of the IMiDs may be preferred.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than 2,000 g/mol. In certain embodiments, the molecular weight of a small molecule is not more than 1,500 g/mol. In certain embodiments, the molecular weight of a small molecule is not more than 1,000 g/mol, not more than 900 g/mol, not more than 800 g/mol, not more than 700 g/mol, not more than 600 g/mol, not more than 500 g/mol, not more than 400 g/mol, not more than 300 g/mol, not more than 200 g/mol, or not more than 100 g/mol. In certain embodiments, the molecular weight of a small molecule is at least 100 g/mol, at least 200 g/mol, at least 300 g/mol, at least 400 g/mol, at least 500 g/mol, at least 600 g/mol, at least 700 g/mol, at least 800 g/mol, or at least 900 g/mol, or at least 1,000 g/mol. Combinations of the above ranges (e.g., at least 200 g/mol and not more than 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “administer,” “administering,” or “administration,” refers to implanting, absorbing, ingesting, injecting, inhaling, or otherwise introducing an IMiD, or a pharmaceutically acceptable salt, solvate, hydrate, polymorph, co-crystal, tautomer, stereoisomer, isotopically labeled derivative, or prodrug thereof to a subject in need thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The terms “condition,” “disease,” and “disorder” are used interchangeably.

An “effective amount” of an IMiD refers to an amount sufficient to elicit the desired biological response, i.e., treating the condition. As will be appreciated by those of ordinary skill in this art, the effective amount of an IMiD may vary depending on such factors as the desired biological endpoint, the pharmacokinetics of the IMiD, the condition being treated, the mode of administration, and the age and health of the subject. An effective amount encompasses therapeutic and prophylactic treatment. For example, in treating cancer, an effective amount of an IMiD may reduce the tumor burden or stop the growth or spread of a tumor.

A “therapeutically effective amount” of an IMiD is an amount sufficient to provide a therapeutic benefit in the treatment of a condition or to delay or minimize one or more symptoms associated with the condition. A therapeutically effective amount of an IMiD means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, autoinflammatory diseases, and autoimmune diseases.

The terms “neoplasm” and “tumor” are used interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal or pathological angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases). In certain embodiments, the angiogenesis is pathological angiogenesis.

A “protein” or “peptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, or synthetic, or any combination of these.

The term “kinase” refers to any enzyme that catalyzes the addition of phosphate groups to an amino acid residue of a substrate (e.g., a protein or nucleoside). For example, a serine kinase catalyzes the addition of a phosphate group to serine residue in a protein. In certain embodiments, the kinase is a tyrosine kinase. Examples of kinases include, but are not limited to, zeta-chain-associated protein kinase-70 (Zap-70), a Janus kinase (e.g., Janus kinase 1 (JAK1), Janus kinase 2 (JAK2), Janus kinase 3 (JAK3), tyrosine kinase 2 (TYK2)), a CMGC kinase (e.g., a cyclin-dependent kinase (CDK, e.g., CDK1, CDK2, CDK2, CDK4, CDK5, CDK7, CDK8, CDK9, CDK10, CDK11, CDK12, CDK13, CDK14, CDK16, CDK20), a mitogen-activated protein kinase (MAPK, e.g., MAPK1, MAPK3, MAPK4, MAPK6, MAPK7, MAPK8, MAPK9, MAPK10, MAPK11, MAPK12, MAPK13, MAPK14, MAPK15), a glycogen synthase kinase 3 (GSK3, e.g., GSK3a, GSK3P), or a CDC-like kinase (CLK, e.g., CLK1, CLK2, CLK3, CLK4)), an AGC kinase (e.g., protein kinase A (PKA), protein kinase C (PKC), protein kinase G (PKG)), a Ca²⁺/calmodulin-dependent protein kinase (CaM kinase, e.g., a specialized CaM kinase, a multifunctional CaM kinase), a casein kinase 1 (CK1, e.g., CK1alpha, CK1beta 1, CK1gamma 1, CK1gamma 2, CK1gamma 3, CK1delta, CK1epsilon), a STE kinase (e.g., a homolog of yeast Sterile 7, Sterile 11, or Sterile 20 kinase), a tyrosine kinase (TK, e.g., a receptor tyrosine kinase (RTK), a non-receptor tyrosine kinase (nRTK)), and a tyrosine-kinase-like kinase (TKL, e.g., a mixed lineage kinase (MLK), RAF, a serine threonine kinase receptor (STKR), a leucine rich repeat kinase (LRRK), a LIM domain kinase (LIMK), a testis expressed serine kinase (TESK), an IL1 receptor associated kinase (IRAK), a receptor interacting protein kinase (RIPK)).

“Zeta-chain-associated protein kinase-70” or “Zap-70” refers to a 70 kDa cytoplasmic protein tyrosine kinase composed of two SH2 domains and a carboxy-terminal kinase domain initiating T-cell responses by the antigen receptor (Wang H., et al., Cold Spring Harb Perspect Biol. 2010, 2, a002279).

### BRIEF DESCRIPTION OF THE DRAWINGS

The accompanying drawings, which constitute a part of this specification, illustrate several embodiments of the invention and together with the description, serve to explain the principles of the invention.

FIG. 1 shows IMiDs induce phosphorylation of Zap-70 in peripheral blood mononuclear cells (PBMCs) and Jurkat cells. (A) PBMCs were cultured with pomalidomide (“Pom”) (0.01-1 μM) for 16 h. Upper panel shows immunoblotting for Zap 70, p-Zap70 and p-LAT. Lower panel shows densitometric analysis of Zap-70. (B) PBMCs were cultured with pomalidomide (0.1 and 1 μM) for the indicated time periods. (C) PBMCs were cultured with lenalidomide (“Len”) (1 μM) for 16 h. (D) Primary T-cells from healthy volunteer were cultured with pomalidomide (0.25-1 μM) for 16 h. (E) Jurkat cells were cultured with pomalidomide (0.5 and 1 μM) for 16 h. Whole cell lysates were subjected to immunoblotting (A-E) using indicated Abs. (F) Jurkat cells were cultured with pomalidomide (0.01-1 μM) or lenalidomide (0.01-1 μM) for 72 h. Cell growth was assessed by MTT assay.

FIG. 2 shows IMiDs bind and activate Zap-70. (A) Jurkat cells were transfected with scrambled (Sc) siRNA or Zap-70 siRNA. Whole cell lysates were subjected to immunoblotting using indicated Abs. (B) Jurkat cells were treated with lenalidomide (1 and 3 M) or pomalidomide (1 and 3 μM) for 4 h and 8 h. Whole cell lysates were subjected to p-Zap ELISA assay. (C) Jurkat whole cell lysates were incubated with pomalidomide-beads in the presence or absence of competitor (1 mM free pomalidomide) for 1 h. After elution, samples were subjected to immunoblotting using anti-Zap-70 Ab. (D) Saturation-transfer difference resulting from the binding of pomalidomide to Zap-70. Pomalidomide is at 320 μM and Zap-70 is approximately 2 μM in deuterated PBS solution. The top spectrum shows the normal 1D spectrum for pomalidomide plus protein, and the bottom spectrum shows the STD. (E) Saturation-transfer difference resulting from the binding of pomalidomide to Zap-70 in the presence of 2.56 mMATP. Pomalidomide is at 320 μM and Zap-70 is approximately 2 μM in deuterated PBS solution. The top spectrum shows the normal 1D spectrum for ATP plus pomalidomide plus protein, and the bottom spectrum shows the STD. Note that both ATP and pomalidomide show binding the Zap-70. (F) Non-cell based Zap-70 kinase assay was carried out, according to manufacturer's protocol.

FIG. 3 shows CRBN expression does not regulate phosphorylation or protein expression of Zap-70. (A) Jurkat cells were transfected with scrambled siRNA (Scsi) or CRBN siRNA (CRBNsi). Whole cell lysates were subjected to immunoblotting using indicated Abs. (B) Jurkat cells transfected with Scsi or CRBNsi were cultured with pomalidomide (1 μM) for 16 h. Whole cell lysates were subjected to immunoblotting using indicated Abs. (C) Jurkat cells were transfected with Scsi or Zap-70 siRNA (Zap-70si) (left panel). The transfectants were further cultured for 72 h, and cell growth was assessed by MTT assay (right panel).

FIG. 4 shows Zap-70 mediates pomalidomide-induced upregulation of NK cell activity. KHYG-1 cells were cultured with pomalidomide (0.25-1 μM) for 24 h. (A) Whole cell lysates were subjected to immunoblotting using indicated Abs. (B) KHYG-1 cells were incubated with calcein-AM-stained U266 cells for 4 h at the indicated effector/target (E/T) ratios. Percent specific lysis was calculated as described previously. (C) KHYG-1 cells were transfected with Scsi or Zap-70si, and then cultured with pomalidomide (0.25 μM) for 72 h in the absence of IL-2. Viable cell number was determined, and cells were then incubated with calcein AM-labeled U266 target cells for 4 h at indicated effector/target (E/T) ratios. Percent specific lysis was calculated as described previously. (D) After transfection with Scsi or Zap-70si, cells were cultured with lenalidomide or pomalidomide for 72 h. Cell growth was assessed by MTT assay. (E) KHYG-1 cells were transfected with scrambled (Scsi) or CRBNsi. The transfectants were then cultured with pomalidomide (0.5 μM) for 24 h, and whole cell lysates were subjected to immunoblotting using indicated Abs. The arrow indicates CRBN expression. (F) Primary NK cells (#1, #2, #3, #4) were isolated from healthy volunteer's PBMCs, as described in Materials and Methods. NK cells were cultured with pomalidomide (0.5 μM) for 24 h, and whole cell lysates were subjected to immunoblotting using indicated Abs. (G) Isolated primary NK cells (#1, #2) were cultured with pomalidomide (left panel: 0.25 and 0.5 μM, right panel: 0.5 and 1 μM) for 24 h, and were then incubated with calcein AM-labeled U266 for 4 h at E/T ratio of 5/1 (left panel) and 10/1 (right panel). Percent specific lysis was calculated as previously described.

FIG. 5 shows pomalidomide upregulates granzyme-B expression via Zap-70. (A) KHYG1 cells were cultured with pomalidomide (0.25-1 μM) for 24 h. (B) Isolated primary NK cells from healthy volunteers (#1, #2) were cultured with pomalidomide (0.3-1 μM) for 24 h. (C) KHYG-1 cells were transfected with Scsi or Zap-70si. After 48 h, cells were cultured for 24 h in the absence or presence (0.5 μM) of pomalidomide. Whole cell lysates and RNAs were subjected to immunoblotting using indicated Abs.

FIG. 6 shows pomalidomide upregulates granzyme-B expression via CRBN. (A) KHYG-1 cells were transfected with Scsi or CRBNsi. After 48 h, cells were cultured in the absence or presence of pomalidomide (0.5 μM) for 24 h, and cell lysates immunoblotted with indicated Abs. (B) KHYG-1 cells were transfected with Scsi or Zap-70si, and then cultured for 72 h with pomalidomide (0.25 μM), in the absence of IL-2. Cells were counted and incubated with calcein AM-labeled U266 for 4 h at indicated effector/target (E/T) ratios.

FIG. 7 shows IKZF3 plays a critical role in pomalidomide-induced GZM-B expression. (A) KHYG-1 cells were transfected with CRBN, IKZF1, or IKZF3 siRNA. The transfectants were then cultured for 24 h with pomalidomide (0.5 μM). The arrow indicates CRBN. (B) KHYG-1 cells were cultured with pomalidomide for 24 h (0.5 μM), in the presence or absence of bortezomib (BTZ; 2.5 and 5 nM). (C, D) KHYG-1 cells were cultured for 24 h with lenalidomide, pomalidomide, or CC-220 (i.e., iberdomide) (0.01-1 μM). Whole cell lysates and RNAs were subjected to immunoblotting using indicated Abs (C) and real-time q-PCR (D), respectively.

FIG. 8 shows ATP and lenalidomide bind to Zap-70. (A) Saturation-transfer difference resulting from the binding of lenalidomide to Zap-70, with lenalidomide (2.56 mM) and Zap-70 (2 μM) in deuterated PBS solution. The top spectrum shows the normal 1D spectrum for lenalidomide plus protein (the protein signals are very small compared to the ligand), and the bottom spectrum shows the STD. (B) Saturation-transfer difference resulting from the binding of lenalidomide to Zap70 in the presence of 2.56 mM ATP, with lenalidomide (2.56 mM) and Zap-70 (2 μM) in deuterated PBS solution. The top spectrum shows the normal 1D spectrum for ATP plus lenalidomide plus protein, and the bottom spectrum shows the STD. These results show that both ATP and lenalidomide bind to Zap-70.

FIG. 9 shows Zap-70 knockdown decreased cytotoxic activity of KHYG-1 cells. (A, B) KHYG-1 cells were transfected with scrambled (Sc) or Zap-70 siRNAs, and then cultured for 72 h with lenalidomide (0.5 μM), in the absence of IL-2 (A) The viable cell number was measured by trypan-blue dye exclusion. (B) KHYG-1 cells were incubated with calcein-AM-stained U266 cells for 4 h at indicated E/T ratios. Percent specific lysis was calculated as described previously. *: p<0.01 compared with Scsi.

FIG. 10 shows pomalidomide maintains primary NK cell viability. Isolated primary NK cells from two healthy volunteers were cultured for 24 h with pomalidomide (0-0.5 μM). Viable cell number was measured by trypan-blue dye exclusion.

FIG. 11 shows GZM-B is transcriptionally regulated by CRBN in KHYG-1 cells. KHYG-1 cells were transfected with scrambled (Sc) or CRBN siRNAs. After 48 h, cells were cultured for 24 h in the absence or presence (0.5 μM) of pomalidomide. RNA was then extracted and subjected to real-time qPCR. Data are representative of three independent experiments, and values are expressed in mean±SD.

FIG. 12 shows Pom induces p-Zap-70 and enhances NK cell activity in NK-92 cells. (A) NK-92 cells were cultured with Pom (0.5 and 1 μM) for 24 h. Whole cell lysates were subjected to immunoblotting using indicated Abs. (B) NK-92 cells were cultured with Pom (0.5 and 1 μM) for 48 h. The cells were subsequently incubated with calcein-AM-stained U266 cells for 4 h at the indicated effector/target (E/T) ratios. Percent specific lysis was calculated as described previously. (C) NK-92 cells were cultured with Pom (0.03-3 μM) for 72 h. Cell growth was assessed by MTT assay. (D) NK-92 cells were transfected with scrambled (Sc) or Zap-70 siRNA (Zap70si). After 72 h incubation, cell viability was measured by trypan-blue dye exclusion. For B, C, and D, data are representative of at least two independent experiments, and values are expressed in mean±SD.

FIG. 13 shows Zap-70 knockdown downregulates GZM-B in NK-92 cells. NK-92 cells were transfected with scrambled (Sc) or Zap-70 siRNA (Zap70). After 72 h incubation, whole cell lysates were subjected to immunoblotting using indicated Abs (upper panel), and the density of bands was assessed by ImageJ software (lower panel).

FIG. 14 shows IKZF3 KD significantly upregulates NK cell activity. KHYG-1 cells were transfected with scrambled (Sc) or IKZF3 siRNAs. After 48 h, cells were further cultured for 24 h in the absence or presence of Porn (0.5 μM). NK cell activity was measured by incubation with calcein AM-labeled U266 cells for 4 h at E/T ratio of 2.5/1. Percent specific lysis was calculated as previously described. Data are representative of two independent experiments, and values are expressed in mean±SD. *: p<0.01.

FIG. 15 shows CC-220 triggers p-Zap70 and enhances NK cell activity in NK-92 cells. (A) NK-92 cells were cultured with Len (1 μM), Pom (0.01, 0.1, 1 μM) or CC-220 (0.01, 0.1, 1 μM) for 24 h. Whole cell lysates were subjected to immunoblotting using indicated Abs. (B) NK-92 cells were cultured with Pom (0.1 and 1 μM) or CC-220 (0.1, and 1 M) for 48 h. NK cell activity was measured by incubation with calcein AM-labeled U266 cells for 4 h at E/T ratio of 2.5/1. Percent specific lysis was calculated as previously described. Data are representative of three independent experiments, and values are expressed in mean±SD. *: p<0.01.

FIG. 16 shows dexamethasone (Dex) suppresses NK cell activity in the presence of Pom. (A) KHYG-1 cells were cultured with Pom (1 μM) in the absence or presence of Dex (250 nM) for 48 h. (B) KHYG-1 cells were cultured with Pom (0.25 and 1 μM) in the absence or presence of Dex (50, 100 and 200 nM) for 48 h. (C) KHYG-1 cells were cultured with Pom (1 μM) for 24 h. The cells were subsequently treated with Dex (25, 50 and 100 nM) for an additional 24 h. NK cell activity was measured by incubation with calcein AM-labeled U266 cells for 4 h at E/T ratio of 2.5/1. Percent specific lysis was calculated as previously described. Data are representative of three independent experiments, and values are expressed in mean±SD. For C, the data was normalized to untreated cells.

### DETAILED DESCRIPTION OF CERTAIN EMBODIMENTS OF THE INVENTION

The present disclosure provides methods of modulating (e.g., inhibiting or increasing) the activity (e.g., aberrant activity, such as increased or decreased activity) of a kinase (e.g., Zap-70). The present disclosure provides methods of modulating (e.g., inhibiting or increasing) the activity (e.g., undesired or aberrant activity, such as increased activity (e.g., activity above normal levels) or decreased activity (e.g., activity below normal levels)), of a kinase in a subject, biological sample, or cell. In certain embodiments, the diseases include proliferative diseases (e.g., cancer (e.g., multiple myeloma)).

In another aspect, the present disclosure provides methods of treating a disease in a subject in need thereof, the method comprising administering to the subject in need thereof an effective amount (e.g., therapeutically effective amount) of an IMiD as described herein.

In another aspect, the present disclosure provides methods of preventing a disease in a subject in need thereof, the method comprising administering to the subject in need thereof an effective amount (e.g., prophylactically effective amount) of an IMiD described herein.

In another aspect, the present disclosure provides methods of increasing the activity of a kinase in a subject in need thereof, the method comprising administering to the subject in need thereof an effective amount of an IMiD.

In certain embodiments, the methods described herein provide an IMiD in an effective amount (e.g., effective for increasing the activity of a kinase, such as Zap-70). In certain embodiments, the effective amount is a therapeutically effective amount. In certain embodiments, a therapeutically effective amount is an amount effective for increasing the activity of a kinase (e.g., Zap-70). In certain embodiments, a therapeutically effective amount is an amount effective for treating a disease (e.g., a disease associated with aberrant activity of a kinase (e.g., a proliferative disease)). In certain embodiments, a therapeutically effective amount is an amount effective for increasing the activity of a kinase and treating a disease (e.g., a disease associated with aberrant activity of a kinase (e.g., a proliferative disease)). In certain embodiments, a therapeutically effective amount is an amount effective for inducing apoptosis in a cell (e.g., malignant cell, premalignant cell). In certain embodiments, a therapeutically effective amount is an amount effective for inducing natural killer cell activity associated with upregulation of granzyme-B (GZM-B) expression.

In certain embodiments, the effective amount is an amount effective for increasing the activity of a kinase by at least 10%, at least 20%, at least 30%, at least 40%, at least 50%, at least 60%, at least 70%, at least 80%, at least 90%, at least 95%, or at least 98%. In certain embodiments, the effective amount is an amount effective for increasing the activity of a kinase by at least 10%, at least 20%, at least 30%, at least 40%, at least 50%, at least 60%, at least 70%, at least 80%, at least 90%, at least 95%, or at least 98%.

In another aspect, the present disclosure provides methods of increasing the activity of a kinase in a biological sample (e.g., an in vitro biological sample), the method comprising contacting the biological sample with an effective amount of an IMiD described herein.

In another aspect, the present disclosure provides methods of increasing the activity of a kinase in a cell (e.g., an in vitro cell), the method comprising contacting the cell with an effective amount of an IMiD described herein.

In certain embodiments, provided are methods of increasing the activity of a kinase (e.g., Zap-70) in a subject, biological sample, or cell by at least about 1%, at least about 3%, at least about 10%, at least about 20%, at least about 30%, at least about 40%, at least about 50%, at least about 60%, at least about 70%, at least about 80%, or at least about 90%. In certain embodiments, the activity of a kinase in a subject, biological sample, or cell is increased by at least about 1%, at least about 3%, at least about 10%, at least about 20%, at least about 30%, at least about 40%, at least about 50%, at least about 60%, at least about 70%, at least about 80%, or at least about 90%. In some embodiments, the activity of a kinase in a subject, biological sample, or cell is selectively increased by the method. In some embodiments, the activity of a kinase (e.g., Zap-70) in a subject, biological sample, or cell is selectively increased by an IMiD.

In certain embodiments, provided are methods for treating a proliferative disease in a subject in need thereof. In certain embodiments, the proliferative disease is cancer (e.g., multiple myeloma). In certain embodiments, the proliferative disease is a solid tumor. In certain embodiments, the proliferative disease is a hematological malignancy.

In certain embodiments, the method described herein superior (e.g., showing improved safety and/or therapeutic effects) or comparable to existing therapy (e.g., chemotherapy).

In certain embodiments, the biological sample or cell (e.g., the biological sample or cell being contacted with an IMiD) is in vitro. In certain embodiments, the biological sample or cell is in vivo. In certain embodiments, the biological sample or cell is ex vivo.

In certain embodiments, the cell is a malignant cell (e.g., cancer cell). In certain embodiments, the cell is a malignant blood cell. In certain embodiments, the cell is a malignant bone marrow cell. In certain embodiments, the cell is an adenocarcinoma cell, blastoma cell, carcinoma cell, or sarcoma cell. In certain embodiments, the cell is a pre-malignant cell (e.g., pre-cancerous cell).

In certain embodiments, the method described herein further comprises administering to the subject in need thereof an additional therapy. In certain embodiments, the additional therapy comprises administering an additional pharmaceutical agent. In certain embodiments, the additional pharmaceutical agent is a small molecule. In certain embodiments, the additional therapy is a cytotoxic chemotherapy (e.g., bortezomib, gemcitabine, cytarabine, daunorubicin, doxorubicin, vincristine, 1-asparaginase, cyclophosphamide, or etoposide). In certain embodiments, the additional therapy is bortezomib. In certain embodiments, the additional pharmaceutical agent is a corticosteroid (e.g., dexamethasone). In certain embodiments, the additional pharmaceutical agent is dexamethasone (Dex).

In certain embodiments, the additional therapy is an epigenetic modifier (e.g., azacitidine or romidepsin). In certain embodiments, the additional therapy is a glucocorticoid. In certain embodiments, the additional therapy is an immunotherapy (e.g., an immunotherapeutic monoclonal antibody). In some embodiments, the additional pharmaceutical agent is bortezomib, and optionally the disease is multiple myeloma.

In certain embodiments, the additional therapy is a cytotoxic chemotherapy, radiation therapy, targeted therapy, hormone therapy, surgery, or stem cell transplantation.

In yet another aspect, the present invention provides IMiDs described herein for use in the treatment of a disease (e.g., a proliferative disease, such as cancer) in a subject in need thereof.

In yet another aspect, the present invention provides IMiDs described herein for use in the prevention of a disease (e.g., a proliferative disease, such as cancer) in a subject in need thereof.

In another aspect, the present disclosure provides IMiDs described herein for use in increasing the activity of a kinase (e.g., Zap-70) in a subject in need thereof.

In another aspect, the present disclosure provides IMiDs described herein for use in increasing the activity of a kinase in a biological sample (e.g., an in vivo or ex vivo biological sample).

In another aspect, the present disclosure provides IMiDs described herein for use in increasing the activity of a kinase in a cell (e.g., an in vivo or ex vivo cell).

In another aspect, the present disclosure provides uses of IMiDs described herein in the manufacture of a medicament for treating a disease in a subject in need thereof.

In another aspect, the present disclosure provides uses of IMiDs described herein in the manufacture of a medicament for preventing a disease in a subject in need thereof.

In certain embodiments, the subject is an animal. The animal may be of either sex and may be at any stage of development. In certain embodiments, the subject described herein is a human (e.g., an adult, juvenile, or child). In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a dog. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the subject is a genetically engineered animal. In certain embodiments, the subject is a transgenic animal (e.g., transgenic mice, transgenic pigs). In certain embodiments, the subject is a fish or reptile.

In certain embodiments, the biological sample or cell (e.g., the biological sample or cell being contacted with an IMiD described herein) is in vitro. In certain embodiments, the biological sample or cell is in vivo or ex vivo. In certain embodiments, the cell is a malignant cell or premalignant cell.

The IMiDs provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the IMiD is suitable for topical administration to the eye of a subject.

The exact amount of an IMiD required to achieve an effective amount will vary from subject to subject, depending, for example, on species, age, and general condition of a subject, severity of the side effects or disorder, identity of the particular IMiD, mode of administration, and the like. An effective amount may be included in a single dose (e.g., single oral dose) or multiple doses (e.g., multiple oral doses). In certain embodiments, when multiple doses are administered to a subject or applied to a biological sample or cell, any two doses of the multiple doses include different or substantially the same amounts of a IMiD described herein. In certain embodiments, when multiple doses are administered to a subject or applied to a biological sample or cell, the frequency of administering the multiple doses to the subject or applying the multiple doses to the biological sample or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample or cell is one dose per day. In certain embodiments, the frequency of administering the multiple doses to the subject or applying the multiple doses to the biological sample or cell is two doses per day. In certain embodiments, the frequency of administering the multiple doses to the subject or applying the multiple doses to the biological sample or cell is three doses per day. In certain embodiments, when multiple doses are administered to a subject or applied to a biological sample or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject or cell. In certain embodiments, the duration between the first dose and last dose of the multiple doses is three months, six months, or one year. In certain embodiments, the duration between the first dose and last dose of the multiple doses is the lifetime of the subject or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an IMiD described herein. In certain embodiments, a dose described herein includes independently between 1 mg and 3 mg, inclusive, of an IMiD described herein. In certain embodiments, a dose described herein includes independently between 3 mg and 10 mg, inclusive, of an IMiD described herein. In certain embodiments, a dose described herein includes independently between 10 mg and 30 mg, inclusive, of an IMiD described herein. In certain embodiments, a dose described herein includes independently between 30 mg and 100 mg, inclusive, of an IMiD described herein.

Dose ranges as described herein provide guidance for the administration of the provided IMiDs to an adult. The amount to be administered to, for example, a child or an adolescent can be determined by a medical practitioner or person skilled in the art and can be lower or the same as that administered to an adult.

An IMiD, as described herein, can be administered in combination with one or more additional pharmaceutical agents (e.g., therapeutically and/or prophylactically active agents). The IMiDs can be administered in combination with additional pharmaceutical agents that improve their activity (e.g., activity (e.g., potency and/or efficacy) in treating a disease in a subject in need thereof, in preventing a disease in a subject in need thereof, in increasing the activity of a kinase (e.g., Zap-70) in a subject, biological sample, or cell), improve bioavailability, improve safety, reduce drug resistance, reduce and/or modify metabolism, inhibit excretion, and/or modify distribution in a subject, biological sample, or cell. It will also be appreciated that the therapy employed may achieve a desired effect for the same disorder, and/or it may achieve different effects. The IMiD can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, cancer, inflammatory disease, autoimmune disease, genetic disease, hematological disease, neurological disease, painful condition, psychiatric disorder, or metabolic disorder) or premalignant condition. Each additional pharmaceutical agent may be administered at a dose and/or on a time schedule determined for that pharmaceutical agent. The additional pharmaceutical agents may also be administered together with each other and/or with the IMiD described herein in a single dose or administered separately in different doses. The particular combination to employ in a regimen will take into account compatibility of the IMiD described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the additional pharmaceutical agent is a chemotherapeutic agent. In certain embodiments, the chemotherapeutic agent is bortezomib.

Also encompassed by the present disclosure are kits (e.g., pharmaceutical packs). In certain embodiments, the kit comprises an IMiD as described herein, and instructions for using the IMiD. In certain embodiments, the kit comprises a first container, wherein the first container includes the IMiD. In some embodiments, the kit further comprises a second container. In certain embodiments, the second container includes an excipient (e.g., an excipient for dilution or suspension of the IMiD). In certain embodiments, the second container includes an additional pharmaceutical agent. In some embodiments, the kit further comprises a third container. In certain embodiments, the third container includes an additional pharmaceutical agent. In some embodiments, the IMiD included in the first container and the excipient or additional pharmaceutical agent included in the second container are combined to form one unit dosage form. In some embodiments, the IMiD included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, or inhaler.

In certain embodiments, the instructions are for administering the IMiD to a subject (e.g., a subject in need of treatment or prevention of a disease described herein). In certain embodiments, the instructions are for contacting a biological sample or cell with the IMiD. In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

The IMiDs, and kits described herein may synergistically increase the activity of a kinase (e.g., Zap-70) induced by the additional pharmaceutical agent(s) in the biological sample or subject. Thus, the combination of the IMiDs or kits with additional pharmaceutical agent(s) may be useful in treating diseases resistant to a treatment using the additional pharmaceutical agent(s) without the IMiDs or kits described herein.

### EXAMPLES

In order that the invention described herein may be more fully understood, the following examples are set forth. The synthetic and biological examples described in this application are offered to illustrate the methods and uses provided herein and are not to be construed in any way as limiting their scope.

### Example 1. Pomalidomide Induced Phosphorylation of Zap-70 in T Cells

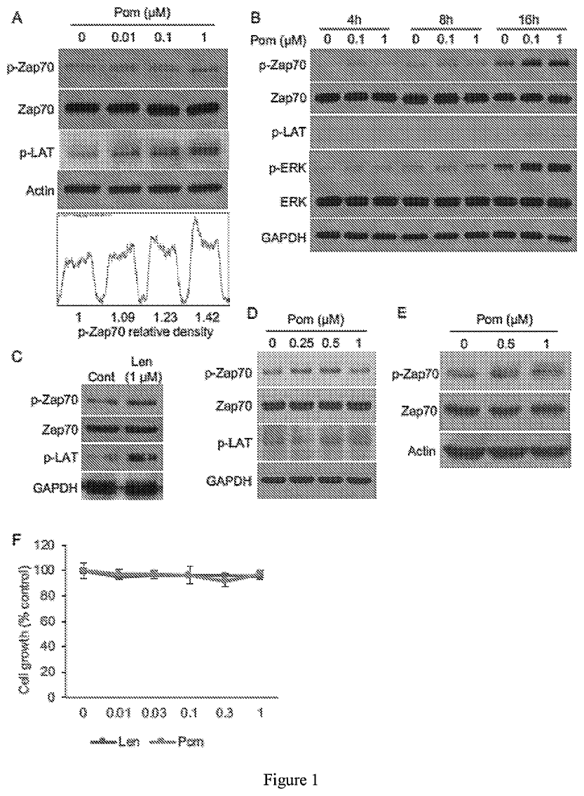

To characterize the effect of IMiDs on Zap-70 function in immune effector cells, pomalidomide triggered phosphorylation of Zap-70 was evaluated along with its known downstream target linker of activated T-cells (LAT) in PBMCs from healthy volunteers. As shown in FIG. 1A (upper panel), pomalidomide induced phosphorylation of both Zap-70 and LAT in PBMCs in a dose-dependent fashion. Image J densitometric analysis confirmed 42% increased p-Zap-70 after pomalidomide (1 μM) treatment (FIG. 1A, lower panel). The increased p-Zap-70, p-LAT, as well as downstream p-ERK in PBMCs triggered by pomalidomide is also time-dependent (FIG. 1B). Of note, lenalidomide similarly triggered p-Zap-70 and p-LAT in PBMCs (FIG. 1C). Since Zap-70 is a mediator of T-cell receptor signaling, whether IMiDs triggered p-Zap-70 in T cells from healthy volunteers was studied. As in PBMCs, pomalidomide treatment induced p-Zap-70 and p-LAT in primary T cells from healthy volunteer (FIG. 1D). pomalidomide similarly induced p-Zap-70 in Jurkat cells in a dose-dependent fashion (FIG. 1E), without altering their proliferation (FIG. 1F).

### Example 2. IMiDs Directly Bind and Activate Zap-70

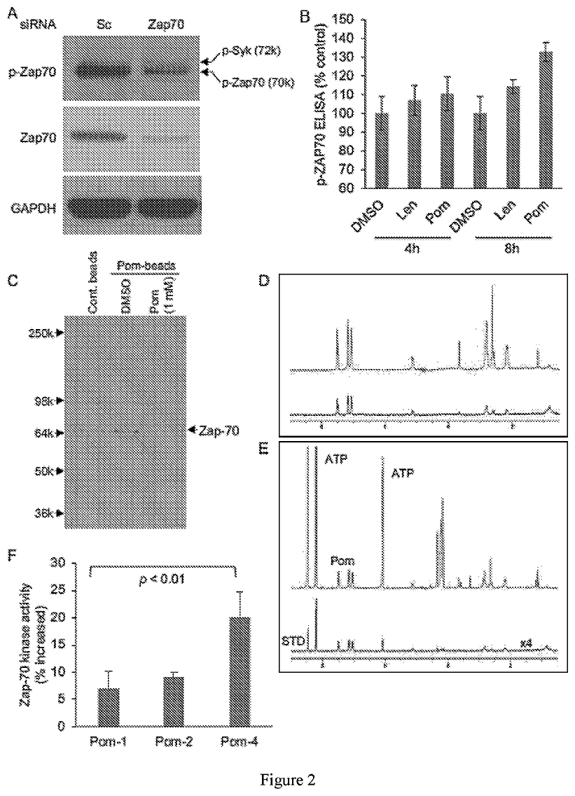

The increased phosphorylation observed by immunoblotting after pomalidomide treatment was p-Zap-70 was also studied, since Ab used for evaluation of p-Zap-70 (Cell Signaling Technology, catalogue #2704) also recognizes p-Syk (spleen tyrosine kinase). Specifically, Zap-70 in Jurkat cells was knocked down, and then immunoblotted cell lysates with p-Zap-70 and Zap-70 Abs; control cells were transfected with scrambled (Sc) siRNA and similarly immunoblotted. The control blot showed 2 bands (upper p-Syk and more prominent lower p-Zap-70), and the lower band was significantly downregulated in Zap-70 knock down cells (FIG. 2A). An ELISA assay to specifically detect p-Zap-70 (Tyr319) in Jurkat cells was also carried out. This assay also showed that both lenalidomide and pomalidomide (Len<Pom) increased p-Zap-70 (Tyr319) in a dose-dependent fashion (FIG. 2B).

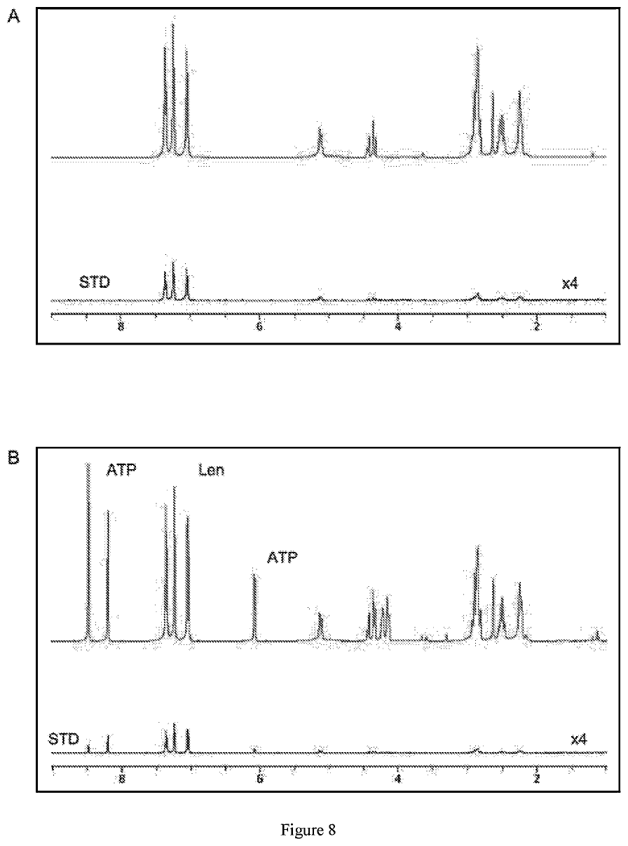

Previous studies have shown that pomalidomide binds not only to CRBN, but also to TP53RK (Hideshima T., et al.; Blood 2000, 96, 2943-2950), thereby inhibiting its function. By immunoblotting, it was demonstrated that Pom-immobilized beads (Pom-beads) pulled down Zap-70, which was inhibited by free pomalidomide (FIG. 2C). Nuclear magnetic resonance (NMR) spectroscopy, as in our prior studies (Hideshima T., et al.; Blood 2000, 96, 2943-2950) was carried out to confirm that pomalidomide directly binds to Zap-70 (FIG. 2D and FIG. 2E). Lenalidomide similarly binds to Zap-70 (FIG. 8A and FIG. 8B). In vitro Zap-70 kinase assay confirmed that IMiDs induced activation of Zap-70 function via phosphorylation (FIG. 2F), consistent with upregulation of downstream p-LAT observed by immunoblotting (FIG. 1A-11D). Taken together, these data show that IMiDs directly bind to Zap-70 and stimulate its activity.

### Example 3. Pomalidomide Induced p-Zap-70 Independent of CRBN

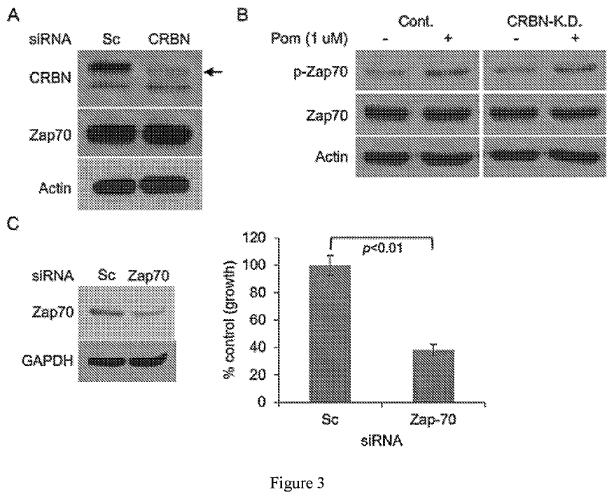

The effect of CRBN on expression of Zap-70 or p-Zap-70 in Jurkat cells was also examined. No significant change in constitutive Zap-70 (FIG. 3A) and p-Zap-70, or in p-Zap-70 induced by pomalidomide (FIG. 3B), in CRBN-knockdown (KD) was observed versus control Sc KD Jurkat cells. To evaluate the biologic role of Zap-70, Zap-70 in Jurkat cells (FIG. 3C, left panel) was knocked down, and observed significant inhibition of their cell growth. (FIG. 3C, right panel). Thus, Zap-70 is a growth factor and independent of CRBN in Jurkat cells.

### Example 4. Zap-70 Mediates Pom-Induced Upregulation of NK Cell Activity

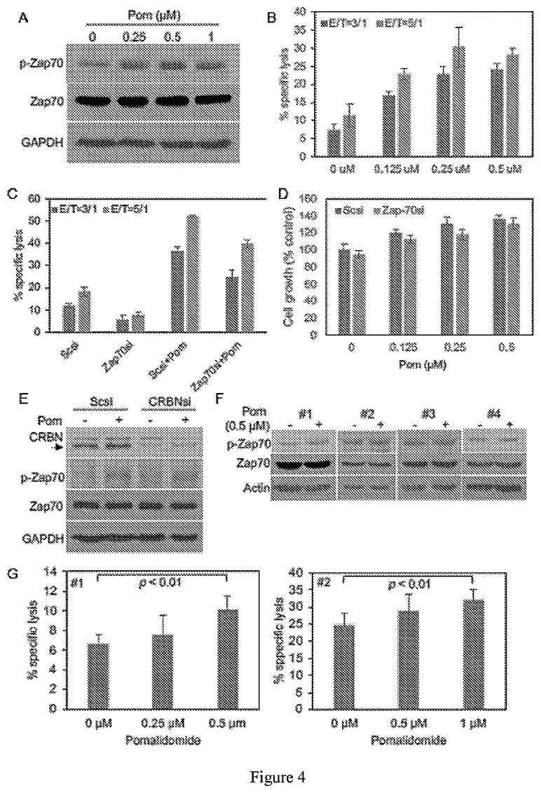

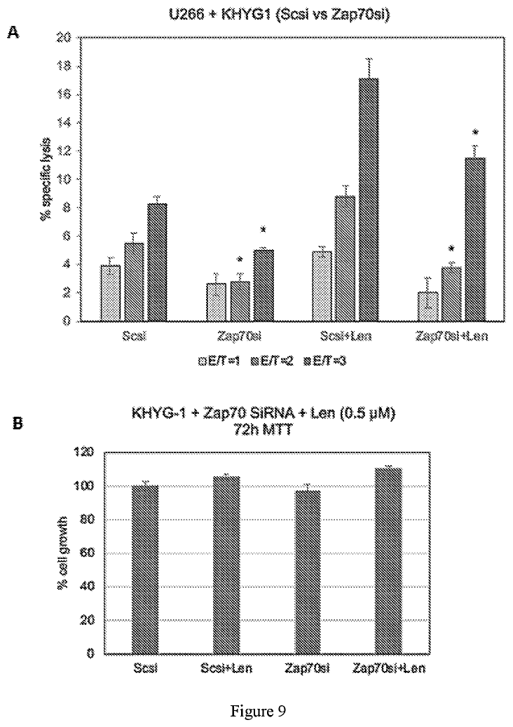

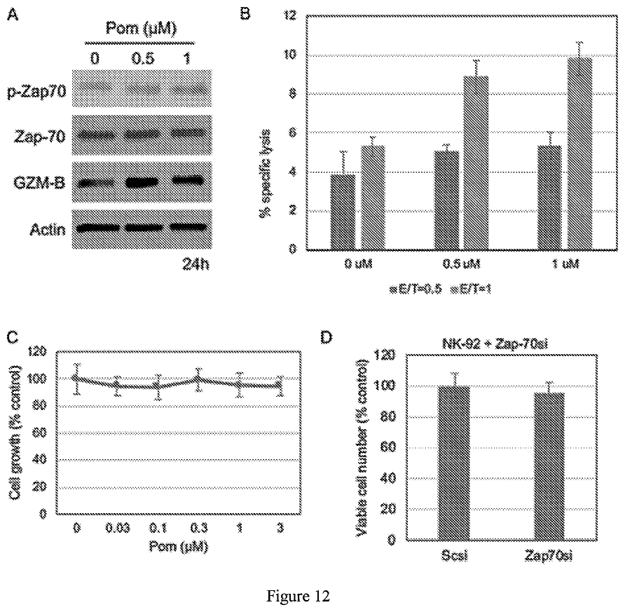

The biological impact of Zap-70 in NK cells using KHYG-1 NK cell line was also validated. Zap-70 is a crucial mediator of T-cell receptor (TCR) signaling (Wang H., et al., Cold Spring Harb Perspect Biol. 2010, 2, a002279); however, its role in NK cells has not yet been delineated. As in PBMCs, Jurkat, or primary T-cells, pomalidomide similarly enhanced p-Zap-70 in KHYG-1 cells (FIG. 4A), and increased their cytotoxicity against U266 cells in a dose-dependent fashion (FIG. 4B). Importantly, Zap-70 KD significantly reduced cytotoxic activity of both Pom-treated (FIG. 4C) and Len-treated KHYG-1 cells (FIG. 9A), without significantly impacting growth (FIG. 4D and FIG. 9B). Finally, as in Jurkat cells (FIGS. 3A and B), CRBN KD in KHYG-1 cells did not alter constitutive Zap-70 protein and p-Zap-70, or Pom-induced p-Zap-70, expression (FIG. 4E). Consistent with KHYG-1 cells, Pom induced increased p-Zap70 and upregulated NK activity. Of note, neither Pom nor Zap-70 KD altered growth in NK-92 cells (FIG. 12).

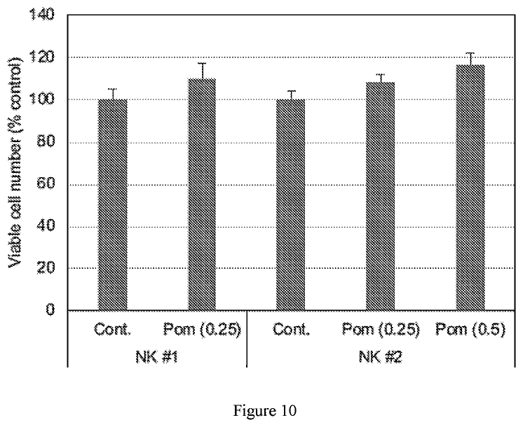

The effect of pomalidomide on p-Zap-70 and NK cell activity was also studied in primary NK cells isolated from healthy volunteers (#1, #2, #3. #4). Importantly and as in KHYG-1 NK cell line, pomalidomide upregulated p-Zap-70 in primary NK cells (FIG. 4F, #1, #2, #3, #4) and significantly enhanced their NK cytolytic activity against U266 cells (FIG. 4G, #1, #2), without significantly effecting NK cell growth. (FIG. 10, #1, #2). These results indicate that Zap-70 mediates, at least in part, constitutive and IMiDs-induced upregulation of NK cell activity.

### Example 5. Pomalidomide Upregulates GZM-B Expression Via Zap-70

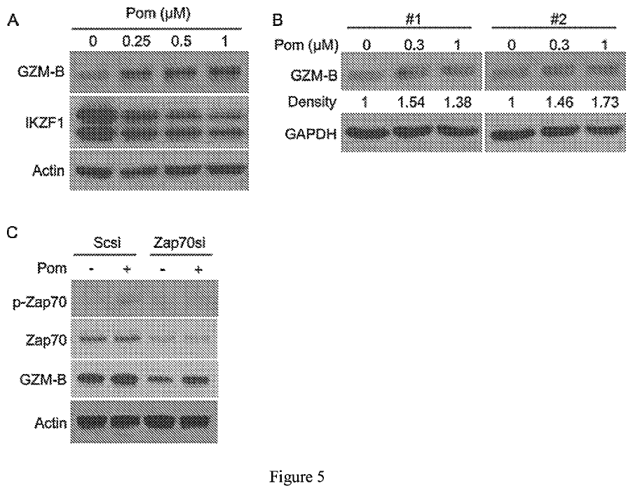

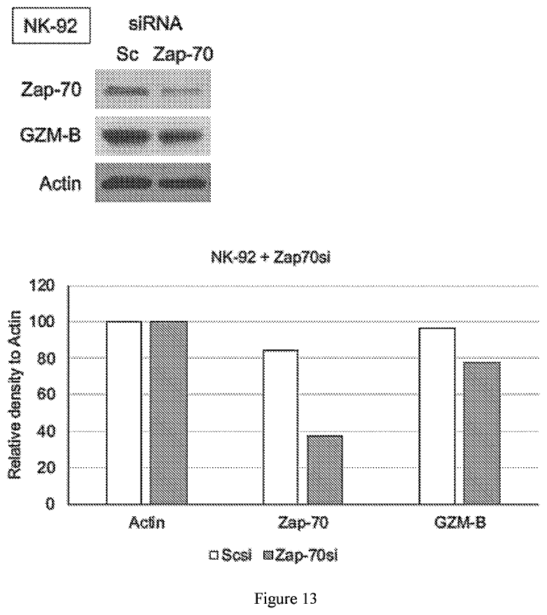

The molecular mechanism whereby IMiDs enhance NK cell activity was also studied. A previous study has demonstrated that lenalidomide upregulates GZM-B expression in MM patient T-cells (Wang H., et al., Cold Spring Harb Perspect Biol. 2010, 2, a002279). Here, it was demonstrated that pomalidomide upregulated GZM-B expression in both KHYG-1 cells (FIG. 5A) and primary NK cells (FIG. 5B, #1, #2) in a dose-dependent fashion. Similar results were observed in NK-92 cells treated with Pom (FIG. 12A). Since Zap-70 KD inhibited Pom-induced upregulation of KHYG-1 cell killing activity (FIG. 4C), whether Zap-70 KD also altered GZM-B expression was also examined. Zap-70 KD decreased both baseline and Pom-induced GZM-B upregulation in KHYG-1 cells (FIG. 5C). Consistent with KHYG-1 cells, we also observed downregulation of GZM-B in NK-92 cells after Zap70 KD (FIG. 13).

### Example 6. Pomalidomide Upregulates GZM-B Expression Via CRBN

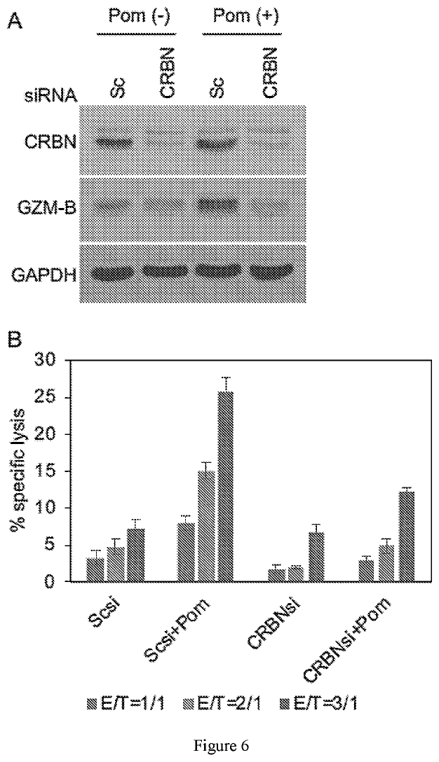

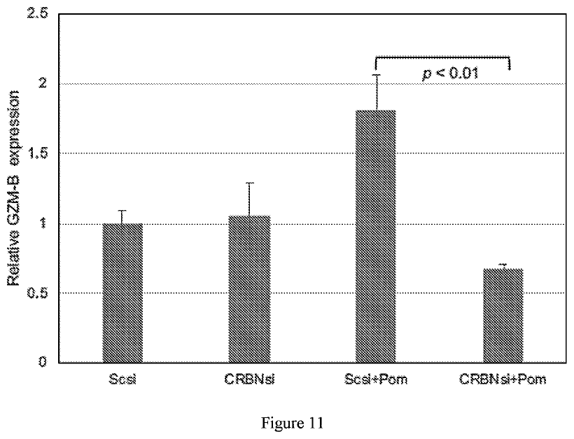

Whether CRBN also mediates Pom-induced GZM-B upregulation in KHYG-1 cells was also studied. Although CRBN KD minimally downregulated constitutive GZM-B expression, it significantly inhibited upregulation of GZM-B triggered by pomalidomide (FIG. 6A). Real-time qPCR confirmed that CRBN transcriptionally regulates GZM-B expression (FIG. 11). Consistent with downregulation of GZM-B, both constitutive and Pom-induced cell killing activity was significantly inhibited in CRBN KD KHYG-1 cells (FIG. 6B). Taken together, these results suggest that Pom-induced enhanced GZM-B and NK cell activity is also mediated, at least in part, by CRBN.

### Example 7. Pomalidomide Upregulates Granzyme-B Expression Via IKZF3

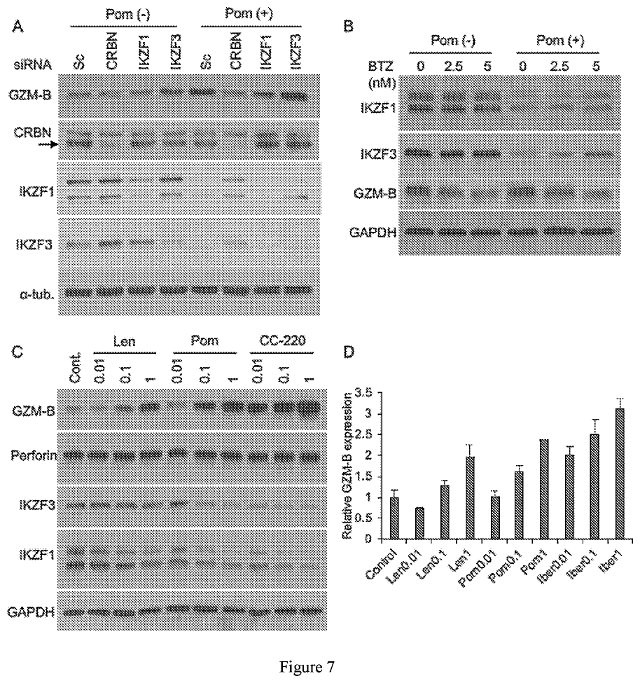

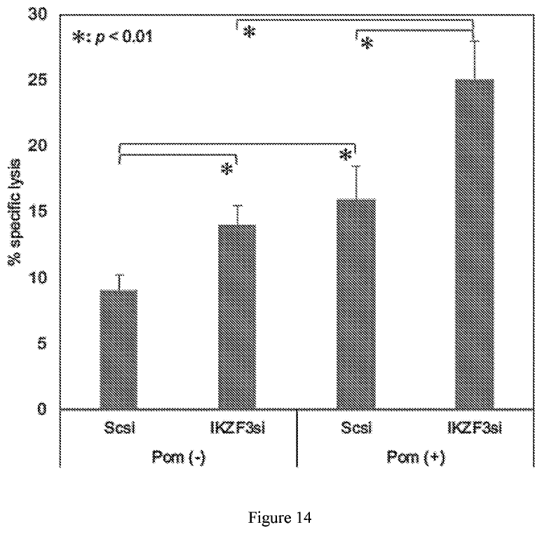

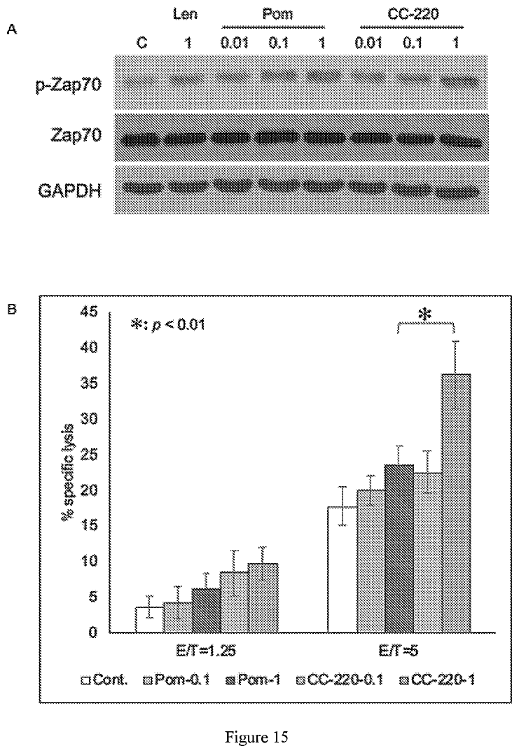

Since IKZF1 and/or IKZF3 are downstream degradation targets of CRBN, their roles in modulating constitutive and Pom-induced GZM-B expression was examined. As in shown in FIG. 7A, IKZF3 KD, but not of IKZF1 KD, enhanced both baseline and Pom-induced GZM-B expression. These results indicate that IKZF3 serves a transcriptional repressor of GZM-B; and conversely, that pomalidomide activation of CRBN E3 ligase and proteasomal degradation of IKZF3 leads to GZM-B upregulation in KHYG-1 cells. IKZF3 KD was also confirmed to significantly upregulated NK cell activity, which is further enhanced in the presence of Pom (FIG. 14). The proteasome inhibitor bortezomib downregulated Pom-induced GZM-B expression in a dose-dependent fashion, associated with upregulation of IKZF3 (FIG. 7B). CC-220 (iberdomide) is a more potent IMiD with enhanced binding affinity to CRBN relative to lenalidomide or pomalidomide, and is now under evaluation in phase 1-2 clinical trials in multiple myeloma. Experiments were conducted to show that CC-220 induced p-Zap-70 in a dose-dependent fashion (FIG. 15A), which was associated with enhanced NK cell activity (FIG. 15B). The potency of lenalidomide, pomalidomide, and CC-220 in triggering GZM-B in KHYG-1 cells was compared. CC-220 more potently upregulated GZM-B than lenalidomide or pomalidomide, which was associated with downregulation of IKZF3 (FIG. 7C). Real-time qPCR of GZM-B further supported this result (FIG. 7D). Of note, none of these IMiDs altered perforin expression, indicating that IMiDs-induced upregulation of NK cell activity is predominantly mediated by GZM-B (FIG. 7C). Taken together, these results show that IMiDs-induced GZM-B upregulation is differentially mediated in NK cells via Zap-70 and via CRBN/IKZF3 pathways.

### Example 8. Pomalidomide Upregulates Granzyme-B Expression Via IKZF3

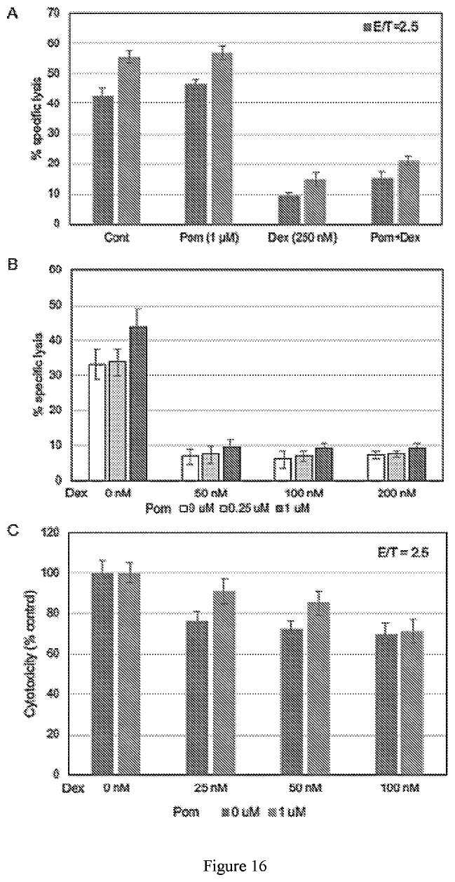

The impact of dexamethasone (Dex) on Pom-induced NK cell activity in KHYG-1 cells was also investigated. It was observed that Dex significantly downregulated NK cell activity, even in the presence of Pom (FIG. 16). This suggests that Dex may have a negative impact on cytotoxic effector cells.

### Example 9. Methods and Materials

Commercial recombinant Zap-70 (Origene, Rockville, MD) was prepared by gel filtration buffer exchange into deuterated phosphate-buffered saline (PBS), which removed any components of the protein storage buffer, including glycerol. Len and Pom were made as d6-DMSO stock solutions and frozen in aliquots prior to use. ATP was stored frozen in aliquots at −20° C. in deuterated PBS. NMR samples were prepared in 5 mm SampleJet tubes to a final volume of 500 μl by adding 5-20 μl of the appropriate IMiD stock solution to the buffer-exchanged protein. The final concentration of protein in the NMR samples was approximately 2 μM. Samples were stored at 6° C. prior to NMR data acquisition. The final concentration in the NMR samples was 320 μM Pom and 2.56 mM Len. ATP was added to a final concentration of 2.56 mM. STD NMR experiments were done with standard methods (3 second protein irradiation as a series of 50 ms selective Gaussian pulses, on/off-resonance RF at 0.82 ppm/−1.0 ppm, respectively). Experiments were run on a Bruker AVANCE III spectrometer (Billerica, MA), operating at 500.13 MHz with a room temperature probe; total acquisition times were approximately 3 h. The STD relies on the fact that a small-molecule ligand that binds to a protein can be saturated indirectly via the protein, and the amount of saturation is related to the strength of the interaction.

Phosphorylation of Zap-70 was evaluated using PathScan Phospho-Zap-70 (Tyr319) Sandwich ELISA Kit (Cell Signaling Technology), according to manufacturer's protocol.

Statistical significance of differences observed in drug-treated versus control cultures was determined using the Wilcoxon signed-ranks test or student t-test. The minimal level of significance was p<0.05.

TABLE 1
Antibody Information

| | | Catalog |
|---|---|---|
| Name of antibody | Vender | number |
| p-Zap70 | Cell Signaling Technology | 2701 |
| Zap70 | Cell Signaling Technology | 2705 |
| p-LAT | Cell Signaling Technology | 3584 |
| p-p44/42 MAPK (ERK1/2) | Cell Signaling Technology | 4376 |
| p44/42 (ERK1/2) | Cell Signaling Technology | 9102 |
| Ikaros (IKZF1) | Cell Signaling Technology | 5443 |
| Granzyme-B (D2H2F) | Cell Signaling Technology | 17215 |
| Aioros (D1C1E) | Cell Signaling Technology | 15103 |
| GAPDH (D4C6R) | Cell Signaling Technology | 97166 |
| Cereblon | Sigma | HPA045910 |
| Perforin 1 (A-2) | Santa Cruz Biotechnology | sc-373943 |
| Beta-actin (C4)-HRP | Santa Cruz Biotechnology | sc-47778 |
| IKZF1 | R&D Systems | AF4984 |

TABLE 2
Sequences of siRNAs

| | Sequence |
|---|---|
| ON-TARGETplus SMARTpool siRNA ZAP-70 | |
| J-005398-17 | GCAACGUCCUGCUGGUUAA |
| J-005398-18 | CCUCAUAGCUGACAUUGAA |
| J-005398-19 | GAACUGUACGCACUCAUGA |
| J-005398-20 | GGAGAUCCCUGUGAGCAAU |
| ON-TARGETplus SMARTpool siRNA CRBN | |
| J-021086-09 | CAAUUAGAAUCCCUCAAUA |
| J-021086-10 | GUAUAAGGCUUGCAACUUG |
| J-021086-11 | GACAUUACCUCUUCAGCUU |
| J-021086-12 | CGACUUCGCUGUGAAUUAG |
| ON-TARGETplus SMARTpool siRNA IKZF1 | |
| J-019092-06 | GCGCAGCGGUCUCAUCUAC |
| J-019092-17 | AGUCAUAUUCUGCGUAGGA |
| J-019092-18 | GCAACGGGCUGUCGGUCAA |
| J-019092-19 | GGUGAUUGUUCAGGUCGAA |
| ON-TARGETplus SMARTpool siRNA IKZF3 | |
| J-006945-05 | GAGCGUGCCUUCUGAGAGA |
| J-006945-06 | GGAGAUGGUUCCAGUUAUC |
| J-006945-07 | AAUCACAUCUAUCAGCAAA |
| J-006945-08 | AGACAUAGGAGAUGAUUCA |

TABLE 3
Primers for real-time qPCR

| Target genes | Directions | Sequences |
|---|---|---|
| Granzyme-B | Forward | AGATGCAACCAATCCTGCTT |
| | Reverse | CATGTCCCCCGATGATCT |
| GAPDH | Forward | GAAGGTGAAGGTCGGAGTCA |
| | Reverse | GGGGTCATTGATGGCAACAATA |

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating a disease in a subject in need thereof, the method comprising administering to the subject in need thereof a therapeutically effective amount of an immunomodulatory imide drug, or a pharmaceutically acceptable salt, solvate, hydrate, polymorph, co-crystal, tautomer, stereoisomer, isotopically labeled derivative, or prodrug thereof.

2. The method of claim 1, wherein the immunomodulatory imide drug, or a pharmaceutically acceptable salt, solvate, hydrate, polymorph, co-crystal, tautomer, stereoisomer, isotopically labeled derivative, or prodrug thereof, is an activator of zeta-chain-associated protein kinase-70.

3. The method of claim 1, wherein the therapeutically effective amount is further effective in increasing the activity of zeta-chain-associated protein kinase-70.

4. The method of claim 1, further comprising administering an additional therapy to the subject in need thereof.

5. (canceled)

6. The method of claim 1, further comprising administering an additional therapeutic agent to the subject in need thereof.

7. The method of claim 6, wherein the additional therapeutic agent is a chemotherapeutic agent.

8. (canceled)

9. The method of claim 6, wherein the additional therapeutic agent is a corticosteroid.

10. (canceled)

11. The method of claim 1, wherein the disease is a proliferative disease.

12. The method of claim 11, wherein the proliferative disease is cancer.

13. The method of claim 11, wherein the proliferative disease is a solid tumor.

14. The method of claim 11, wherein the proliferative disease is a hematological malignancy.

15. (canceled)

16. A method of increasing the activity of a zeta-chain-associated protein kinase-70 in a subject in need thereof, the method comprising administering to the subject in need thereof an effective amount of an immunomodulatory imide drug, or a pharmaceutically acceptable salt, solvate, hydrate, polymorph, co-crystal, tautomer, stereoisomer, isotopically labeled derivative, or prodrug thereof.

17. The method of claim 16, wherein the method comprises inducing natural killer cell activity in a subject in need thereof.

18. The method of claim 17, wherein the immunomodulatory imide drug-induced natural killer cell activity is associated with upregulation of granzyme-B (GZM-B) expression.

19. (canceled)

20. (canceled)

21. A method of increasing the activity of a zeta-chain-associated protein kinase-70 in a biological sample or cell, the method comprising contacting the biological sample or cell with an effective amount of an immunomodulatory imide drug, or a pharmaceutically acceptable salt, solvate, hydrate, polymorph, co-crystal, tautomer, stereoisomer, isotopically labeled derivative, or prodrug thereof.

22. A method of enhancing natural killer cell activity in a biological sample or cell, the method comprising contacting the biological sample or cell with an effective amount of an immunomodulatory imide drug, or a pharmaceutically acceptable salt, solvate, hydrate, polymorph, co-crystal, tautomer, stereoisomer, isotopically labeled derivative, or prodrug thereof.

23. The method of claim 21, wherein the biological sample or cell is in vitro.

24. (canceled)

25. A kit comprising:
an immunomodulatory drug, or a pharmaceutically acceptable salt, solvate, hydrate, polymorph, co-crystal, tautomer, stereoisomer, isotopically labeled derivative, or prodrug thereof; and
instructions for using the immunomodulatory drug, or a pharmaceutically acceptable salt, solvate, hydrate, polymorph, co-crystal, tautomer, stereoisomer, isotopically labeled derivative, or prodrug thereof.

26. The method of claim 1, wherein the immunomodulatory imide drug is selected from the group consisting of thalidomide, lenalidomide, pomalidomide, and iberdomide.

27. (canceled)

28. (canceled)

29. The method of claim 22, wherein the biological sample or cell is in vitro.
